(19) United States
(12) Patent Application Publication
(10) Pub. No.: US 2026/0227390 A1
(43) Pub. Date: Aug. 6, 2026

(54) SYSTEM AND METHOD FOR ANALYZING ANTIBODY CO-FORMULATIONS

(71) Applicant: AMGEN INC., Thousand Oaks, CA (US)

(72) Inventors: Aaron Daniel MILLER, Simi Valley, CA (US); Rupa PADAKI, Moorpark, CA (US); Scott Thomas KUHNS, Newbury Park, CA (US); Jette WYPYCH, Calabasas, CA (US)

(73) Assignee: AMGEN INC., Thousand Oaks, CA (US)

(21) Appl. No.: 19/151,407

(22) PCT Filed: Jan. 29, 2024
(86) PCT No.: PCT/US2024/013284

§ 371 Date: Jul. 28, 2025

Related U.S. Application Data
(60) Provisional application No. 63/482,156, filed on Jan. 30, 2023.

(51) Int. Cl. G01N 33/543; C07K 16/42; G01N 33/563; G01N 33/564; G01N 33/68

(52) U.S. Cl. CPC G01N 33/54313; C07K 16/4208; G01N 33/563; G01N 33/564; G01N 33/6857; G01N 33/686

(57) ABSTRACT

The disclosure provides systems and methods for detecting and quantifying two or more different antigen-binding proteins in a sample. The systems involve donor beads, acceptor beads, and conjugate proteins, which may be incubated with the sample to quantify the antigen-binding proteins via a proximity-based homogenous assay.

### FIELD

The disclosure relates to systems and methods for detecting and quantifying two or more antigen-binding proteins (e.g., antibodies) that are co-formulated in a single composition.

### BACKGROUND

Combination therapy using two or more molecules with complimentary pharmacological effects has led to an increased interest in the development of coformulation products. Co-formulations or fixed-dose combination drugs (FDCs) are therapeutics in which two or more separate drug components (e.g., a small molecule and biologic, or two different biologics such as therapeutic antibodies) are combined in a single dosage form. These products can often reduce the number and volume of injections, improve patient compliance, and reduce discomfort.

As co-formulated therapeutics are classified as new molecular entities (NMEs), they are subject to clinical evaluation by regulatory bodies, such as the U.S. Food and Drug Administration (FDA). Thus, additional co-formulating existing therapeutics may be subject to further analysis and evaluation, even though the efficacy and safety of each individual therapeutic has already been established through independent clinical trials.

There are also many chemistry, manufacturing, and controls issues that need to be addressed for a combination biologic. These include analytical challenges in characterization of each molecule in the co-formulation, manufacturing issues of formulating higher concentration biologics, and stability problems such as protein-protein interactions, protein aggregation, and subvisible particle formulation.

There remains a need for systems and methods for accurately determining the concentrations of co-formulated biologic molecules in a pharmaceutical preparation or sample.

### BRIEF SUMMARY

The disclosure provides a system for detecting and quantifying two or more different antigen-binding proteins in a sample, which system comprises: (a) donor beads, wherein each donor bead comprises a photosensitizer and a first binding pair member; (b) acceptor beads, wherein each acceptor bead comprises a chemiluminescent dye and an anti-idiotype antibody that specifically binds to either of the two or more different antigen-binding proteins and; (c) conjugate proteins, wherein each conjugate protein comprises either of the two or more different antigen-binding proteins comprising a second binding pair member, and wherein the first binding pair member is configured to specifically bind to the second binding pair member. By way of example, a donor bead may be conjugated to the photosensitizer and/or first binding pair member. By way of example, an acceptor bead may be conjugated to the chemiluminescent dye and/or anti-idiotype antibody. By way of example, the antigen-binding protein of the conjugate protein may be conjugated to the second binding pair member.

In some aspects of the system, each of the antigen-binding proteins is an antibody, an antibody fragment, or a bispecific T cell engager (BiTE®) molecule.

In some aspects of the system, the photosensitizer comprises phthalocyanine.

In some aspects of the system, the first binding pair member comprises streptavidin, glutathione S-transferase (GST), or an affinity tag peptide such as a hexahistidine or a FLAG peptide.

In some aspects of the system, the second binding pair member comprises biotin, glutathione, or anti-affinity tag antibody such as an anti-hexahistidine antibody or an anti-FLAG antibody.

In some aspects of the system, the first binding pair member comprises streptavidin and the second binding pair member comprises biotin.

In some embodiments, the system comprises (i) a first acceptor bead comprising (e.g., conjugated to) a first chemiluminescent dye and a first anti-idiotype antibody that binds to a first antigen-binding protein in the sample and (ii) a second acceptor bead comprising (e.g., conjugated to) a second chemiluminescent dye and a second anti-idiotype antibody that binds to a second antigen-binding protein in the sample. In some aspects, the first chemiluminescent dye comprises terbium (Tb) and the second chemiluminescent dye comprises europium (Eu). In some aspects, the first antigen-binding protein is a first therapeutic antibody and the second antigen-binding protein is a second therapeutic antibody. In some aspects, the system comprises a first conjugate protein comprising the first antigen-binding protein and biotin and a second conjugate protein comprising the second antigen-binding protein and biotin. By way of example, the first antigen-binding protein may be conjugated to the biotin, and/or the second antigen-binding protein may be conjugated to the biotin.

In some aspects of the system, the sample is a composition comprising the two or more different antigen-binding proteins and a pharmaceutically acceptable carrier.

The disclosure also provides a method of determining a presence, absence, or concentration of two or more different antigen-binding proteins in a sample, which method comprises: (1) incubating the sample with the above-described system; (2) exposing the incubated sample to light excitation; and (3) detecting at least one chemiluminescent signal following light excitation and comparing the detected chemiluminescent signal intensity to a predetermined reference value. The at least one detected chemiluminescent signal is inversely proportional to the concentration each of the two or more different antigen-binding proteins. Accordingly, the presence, absence, or concentration of two or more different antigen-binding proteins is detected.

In some aspects of the method, the predetermined reference value comprises a threshold value corresponding to a concentration of each of the antigen-binding proteins, and wherein the detected chemiluminescent signal greater than the threshold value indicates that the sample lacks said antigen-binding protein or comprises said antigen-binding protein below the concentration, and a chemiluminescent signal less than or equal to the threshold value indicates that the sample comprises the antigen-binding protein at a concentration greater than or equal to the concentration.

In some aspects of the method, the light excitation wavelength is about 500 nm to about 800 nm, such as about 680 nm.

In some aspects of the method, the sample is a composition comprising the two or more different antigen-binding proteins and a pharmaceutically acceptable carrier.

In some aspects of the method, each of the antigen-binding proteins is an antibody, an antibody fragment, or a bispecific T cell engager (BiTE®) molecule.

In some aspects of the method, the system comprises (i) a first acceptor bead comprising (e.g., conjugated to) a first chemiluminescent dye and a first anti-idiotype antibody that binds to a first antigen-binding protein in the sample and (ii) a second acceptor bead comprising (e.g., conjugated to) a second chemiluminescent dye and a second anti-idiotype antibody that binds to a second antigen-binding protein in the sample.

In some aspects of the method, the first antigen-binding protein comprises a first therapeutic antibody and the second antigen-binding protein comprises a second therapeutic antibody.

In some aspects, the method comprises detecting a first signal emitted from the first chemiluminescent dye and a second signal emitted from the second chemiluminescent dye.

In some aspects of the method, the first signal and second signal comprise two different wavelengths.

In some aspects of the method, the wavelength of the first signal is about 520 nm to about 620 nm and the wavelength of the second signal is about 520 nm to about 620 nm.

In some aspects of the method, the wavelength of the first signal is about 545 nm and the wavelength of the second signal is about 615 nm.

In some aspects, the method further comprises quantifying the two or more antigen-binding proteins in the sample.

In some aspects of the method, the reference value is from a calibration curve. The calibration curve may be for each acceptor bead of the system.

In some aspects, the method further comprises generating a calibration curve for each acceptor bead of the system.

### BRIEF DESCRIPTION OF THE DRAWINGS

FIG. 1 is a schematic diagram illustrating an embodiment of the method described herein.

FIGS. 2A and 2B are graphs showing dose-dependent responses of a bead-based assay for Ab1 (FIG. 2A) and Ab2 (FIG. 2B) as described in the Example.

FIGS. 3A and 3B are graphs showing that Ab1 (FIG. 3A) and Ab2 (FIG. 3B) in co-formulation showed percent relative binding comparable to their respective controls.

FIG. 4 shows the linearity of the no-wash assay described in the Example with respect to Ab1 in co-formulation with Ab2. Varying concentrations of Ab1 were co-formulated with 10 mg/mL Ab2.

FIGS. 5A and 5B are graphs showing the Ab1- and Ab2-specific reagents used in the coformulation assay described in the Example demonstrate high specificity with low potential for cross-reactivity of reagents.

FIGS. 6A and 6B are graphs showing the Terbium (FIG. 6A) and Europium (FIG. 6B) signals obtained in the mock coformulation assay described in the Example.

### DETAILED DESCRIPTION

The present disclosure is predicated, at least in part, on the development of a homogenous, multiplex assay to simultaneously detect and quantify biomolecules (e.g., antibodies) co-formulated in a single sample. The no-wash assay described herein is adaptable to different co-formulations and scalable to larger formats.

In some embodiments, the disclosure provides a bead-based system for detecting and quantifying two or more different antigen-binding proteins in a sample. Upon a biological interaction that brings the beads in close proximity (i.e., ≤200 nm), a cascade of chemical reactions occurs to produce a greatly amplified signal. On light excitation, a photosensitizer (e.g., phthalocyanine) in a “donor” bead converts ambient oxygen to a more excited singlet state at a rate of nearly 60,000 oxygen singlets per second (see, e.g., Eglen, R. M. et. al., Curr Chem Genomics, 1: 2-10 (2008) PMID 20161822). The singlet state oxygen molecules diffuse across to react initially with a thioxene chemical dye in an “acceptor” bead, resulting in a resonance energy transfer to anthracene and then rubrene chemical dyes, with this trio of chemical dyes referred to as the “TAR” (thioxene, anthracene, rubene) complex (see, e.g., US 20040175696A1). In some embodiments, rubrene is the final chemiluminescent dye that emits signal at a distinct wavelength. In other embodiments, a final resonance energy transfer occurs within the same bead to either terbium (Tb), europium (Eu), or samarium (Sm) chemiluminescent dyes that emit at distinct detectable wavelengths (e.g., 520-620 nm). Such Amplified Luminescent Proximity Homogeneous Assay (ALPHA) systems have been used to detect and measure protein and small molecule analytes in biological samples. The capability of having the final chemiluminescent dye in each unique acceptor bead emit luminescent signal at distinct wavelengths with limited spectral overlap allows complimentary acceptor bead pairs (e.g., Tb and Eu Acceptor Beads) to be used in multiplex assays to analyze distinct molecules and binding events, including but not limited to small and large biomolecules or analytes, in single-plate homogeneous no-wash assays.

The present application describes for the first time a no-wash screening system, such as an ALPHA multiplex system, employing anti-idiotype antibodies to detect and/or quantify two or more co-formulated antigen-binding proteins. The system comprises: (a) donor beads, wherein each donor bead comprises (e.g., is conjugated to) a photosensitizer and a first binding pair member; (b) acceptor beads, wherein each acceptor bead comprises (e.g., is conjugated to) a chemiluminescent dye and an anti-idiotype antibody that binds to either of the two or more different antigen-binding proteins and; (c) conjugate proteins, wherein each conjugate protein comprises either of the two or more different antigen-binding proteins and a second binding pair member (for example, conjugated to the antigen-binding protein). Also described herein are methods of using the aforementioned system to detect and/or quantify two or more different antigen-binding proteins in a sample.

### Antigen-Binding Proteins

The term “antigen-binding protein,” as used herein, refers to a proteinaceous molecule that specifically binds to an antigen. For example, an antigen-binding protein may comprise an antibody or an antigen-binding fragment thereof, (such as a monoclonal antibody, for example an IgG1 monoclonal antibody), an antibody protein product, a bi-specific T cell engager (BiTE®) molecule, a bispecific antibody, a trispecific antibody, or an Fc fusion protein.

An antigen-binding protein typically comprises the heavy chain variable region (VH) and/or the light chain variable region (VL) of an antibody, or comprises domains derived therefrom. In some embodiments, an antigen-binding protein comprises the structural requirements of an antibody which are sufficient for immunospecific target binding. This structural requirement may be defined by, for example, the presence of at least three light chain complementarity determining regions (CDRs) (i.e., CDR1, CDR2 and CDR3 of the VL region) and/or three heavy chain CDRs (i.e., CDR1, CDR2 and CDR3 of the VH region), or of all six CDRs. It is within the knowledge of a skilled person where (and in which order) those CDRs are located in the antigen-binding protein.

As used herein, the term “antibody” refers to an immunoglobulin of any isotype with specific binding to the target antigen; an antibody may be a polyclonal or monoclonal antibody, a chimeric antibody, a humanized antibody, a human antibody, etc. In a native antibody, a heavy chain comprises a variable region, VH, and three constant regions, CH1, CH2, and CH3. The VH domain is at the amino-terminus of the heavy chain, and the CH3 domain is at the carboxy-terminus. In a native antibody, a light chain comprises a variable region, VL, and a constant region, CL. The variable region of the light chain is at the amino-terminus of the light chain. In a native antibody, the variable regions of each light/heavy chain pair typically form the antigen-binding site. The constant regions are typically responsible for effector function.

In a human antibody, CH1 means a region having the amino acid sequence at positions 118 to 215 of the EU index or EU numbering system, which is based on the sequential numbering of the first human IgG1 sequenced (i.e., the “EU antibody”) (Edelman et al., Proc Natl Acad Sci USA, 63(1): 78-85 (1969)). A highly flexible amino acid region called a “hinge region” exists between CH1 and CH2. CH2 represents a region having the amino acid sequence at positions 231 to 340 of the EU index, and CH3 represents a region having the amino acid sequence at positions 341 to 446 of the EU index.

“CL” represents a constant region of a light chain. In the case of a κ chain of a human antibody, CL represents a region having the amino acid sequence at positions 108 to 214 of the EU index. In a λ chain, CL represents a region having the amino acid sequence at positions 108 to 215.

In a native antibody, the variable regions typically exhibit the same general structure in which relatively conserved framework regions (FRs) are joined by three hypervariable CDRs. The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and FR4. Typically, CDR3 is the greatest source of molecular diversity within the antigen binding site. The assignment of amino acids to each domain is typically in accordance with the definitions of Kabat et al. (1991) Sequences of Proteins of Immunological Interest (National Institutes of Health, Publication No. 91-3242, vols. 1-3, Bethesda, Md.); Chothia, C., and Lesk, A. M. (1987) J. Mol. Biol., 196: 901-917; or Chothia C. et al., Nature, 342:878-883 (1989). In some embodiments, the CDRs of an antigen binding protein are defined according to the definition of Kabat or Chothia. In the present application, the term “CDR” refers to a CDR from either the light or heavy chain, unless otherwise specified.

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4.

The antibody can be a monoclonal antibody or a polyclonal antibody. The term “monoclonal antibody,” as used herein, refers to an antibody produced by a single clone of B lymphocytes that is directed against a single epitope on an antigen. Monoclonal antibodies typically are produced using hybridoma technology, as first described in Kohler and Milstein, Eur. J. Immunol., 5: 511-519 (1976). Monoclonal antibodies may also be produced using recombinant DNA methods (see, e.g., U.S. Pat. No. 4,816,567), isolated from phage display antibody libraries (see, e.g., Clackson et al. Nature, 352: 624-628 (1991)); and Marks et al., J. Mol. Biol., 222: 581-597 (1991)), or produced from transgenic mice carrying a fully human immunoglobulin system (see, e.g., XENOMOUSE™ mouse, Green et al. (1994) Nature Genetics 7:13-21, US 2003-0070185, WO 96/34096, and WO 96/33735). In contrast, “polyclonal” antibodies are antibodies that are secreted by different B cell lineages within an animal. Polyclonal antibodies are a collection of immunoglobulin molecules that recognize multiple epitopes on the same antigen.

The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence.

An antibody can be cleaved into fragments by enzymes, e.g., papain, pepsin, or other engineered site-specific proteases (such as those commercially available from Genovis A B, Lund, Sweden). Papain cleaves an antibody to produce two Fab fragments and a single Fc fragment. Pepsin cleaves an antibody to produce a F(ab′)2 fragment and a pFc′ fragment. In exemplary aspects, the antigen-binding protein of the present disclosure comprises an antigen binding antibody fragment. As used herein, the term “antigen binding antibody fragment” refers to a portion of an antibody molecule that is capable of binding to the antigen of the antibody and is also known as “antigen-binding fragment” or “antigen-binding portion.” In exemplary instances, the antigen binding antibody fragment is a Fab fragment or a F(ab′)2 fragment.

The architecture of antibodies has been exploited to create a growing range of alternative formats that span a molecular-weight range of at least about 12-150 kDa and has a valency (n) range from monomeric (n=1), to dimeric (n=2), to trimeric (n=3), to tetrameric (n=4), and potentially higher; such alternative formats are referred to herein as “antibody protein products.” Antibody protein products include those based on the full antibody structure and those that mimic antibody fragments which retain full antigen-binding capacity, e.g., scFvs, Fabs (e.g., Fab, Fab′, and F(ab′)₂) and VHH/VH. The smallest antigen binding antibody fragment that retains its complete antigen binding site is the Fv fragment, which consists entirely of variable (V) regions of the light and heavy chains. A soluble, flexible amino acid peptide linker is used to connect the V regions in a scFv (single chain fragment variable) fragment for stabilization of the molecule, or the constant (C) domains are added to the V regions to generate a Fab fragment. Both scFv and Fab fragments can be easily produced in host cells, e.g., prokaryotic host cells. A VHH/VH (or nanobody) is the antigen binding fragment of heavy chain only antibodies. Heavy chain only antibodies (HcAb) are naturally produced by camelids and sharks. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. A peptibody or peptide-Fc fusion is yet another antibody protein product. The structure of a peptibody consists of a biologically active peptide grafted onto an Fc domain (see, e.g., Shimamoto et al., mAbs 4(5): 586-591 (2012)).

The antigen-binding protein of the present disclosure may comprise any one of the above-described antibody protein products. In exemplary aspects, the antigen-binding protein of the present disclosure may comprise any one of an scFv, Fab VHH/VH, Fv fragment, ds-scFv, scFab, dimeric antibody, multimeric antibody (e.g., a diabody, triabody, tetrabody), miniAb, peptibody VHH/VH of camelid heavy chain antibody, sdAb, diabody; a triabody; a tetrabody; a bispecific or trispecific antibody, BsIgG, appended IgG, BsAb fragment, bispecific fusion protein, or BsAb conjugate.

In certain aspects, the antigen-binding proteins of the present disclosure may be “bispecific,” meaning that they are capable of specifically binding to two different antigens. In another aspect, the antigen-binding proteins of the present disclosure may be “trispecific,” meaning that they are capable of specifically binding to three different antigens. In another aspect, the antigen-binding proteins of the present disclosure may be “tetraspecific,” meaning that they are capable of specifically binding to four different antigens.

In some embodiments, the antigen-binding protein is a BiTE® molecule. BiTE® molecules are engineered bispecific antigen binding constructs which direct the cytotoxic activity of T cells against cancer cells. They are the fusion of two single-chain variable fragments (scFvs) of different antibodies, or amino acid sequences from four different genes, on a single peptide chain of about 55 kilodaltons. One of the scFvs binds to T cells via the CD3 receptor, and the other to a tumor cell via a tumor specific molecule. Blinatumomab (BLINCYTO® product) is an example of a BiTE® molecule, specific for CD19. BiTE® molecules that are modified, such as those modified to extend their half-lives, can also be used in the disclosed methods. By their design, BiTE® molecules are uniquely suited to transiently connect T cells with target cells and, at the same time, potently activate the inherent cytolytic potential of T cells against target cells. See e.g., WO 99/54440, WO 2005/040220, and WO 2008/119567.

In certain embodiments of the disclosure, the antigen-binding proteins may be multivalent. The valency of the binding protein denotes the number of individual antigen binding domains within the binding protein. In some embodiments, a bispecific antigen binding protein may be multivalent. For instance, in certain embodiments, a bispecific antigen binding protein may be tetravalent by comprising four antigen-binding domains: two antigen-binding domains binding to a first target antigen and two antigen-binding domains binding to a second target antigen.

As used herein, the terms “antigen binding domain” and “binding domain” may be used interchangeably to refer to the region of the antigen-binding protein that contains the amino acid residues that interact with the antigen and confer on the antigen-binding protein its specificity and affinity for the antigen. In some embodiments, the binding domain may be derived from the natural ligands of the target antigen(s). As used herein, the term “target antigen(s)” refers to a first target antigen and/or a second target antigen of a bispecific molecule and also refers to a first target antigen, a second target antigen, a third target antigen, and/or a fourth target antigen of a tetraspecific molecule.

An antigen-binding protein may comprise an immunoglobulin domain. The term “immunoglobulin domain,” as used herein, refers to a peptide comprising an amino acid sequence similar to that of immunoglobulin (i.e., antibody) and comprising approximately 100 amino acid residues including at least two cysteine residues. Examples of immunoglobulin domains include VH, CH1, CH2, and CH3 of an antibody heavy chain, and VL and CL of an antibody light chain. In addition, the immunoglobulin domain is found in proteins other than immunoglobulin. Examples of the immunoglobulin domain in proteins other than immunoglobulin include an immunoglobulin domain included in a protein belonging to an immunoglobulin super family, such as a major histocompatibility complex (MHC), CD1, B7, T-cell receptor (TCR), and the like.

In some embodiments, each of the two or more antigen-binding proteins are therapeutic proteins. As used herein “therapeutic protein,” and variations of this root term, has its ordinary and customary meaning as would be understood by one of ordinary skill in the art in view of this disclosure. It refers to a polypeptide for medical use in a subject, typically a human subject. For example, each of the two or more antigen-binding proteins may be therapeutic antigen binding proteins such as therapeutic antibodies. By way of example, a therapeutic protein may be a polypeptide approved for medical use by a government regulatory authority, such as the Food and Drug Administration or the European Medicines Agency.

Therapeutic antigen-binding proteins, such as antibodies, encompassed by the present disclosure may include polypeptides that bind to one or more of the following: (i) CD proteins, including CD3, CD4, CD8, CD19, CD20, CD22, CD30, and CD34; including those that interfere with receptor binding; (ii) HER receptor family proteins, including HER2, HER3, HER4, and the EGF receptor; (iii) cell adhesion molecules, for example, LFA-I, MoI, pl50, 95, VLA-4, ICAM-I, VCAM, and alpha v/beta 3 integrin; (iv) growth factors, such as vascular endothelial growth factor (“VEGF”), growth hormone, thyroid stimulating hormone, follicle stimulating hormone, luteinizing hormone, growth hormone releasing factor, parathyroid hormone, Mullerian-inhibiting substance, human macrophage inflammatory protein (MIP-1alpha), erythropoietin (EPO), nerve growth factor, such as NGF-beta, platelet-derived growth factor (PDGF), fibroblast growth factors, including, for instance, aFGF and bFGF, epidermal growth factor (EGF), transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des(1-3)-IGF-I (brain IGF-I), and osteoinductive factors; (v) insulins and insulin-related proteins, including insulin, insulin A-chain, insulin B-chain, proinsulin, and insulin-like growth factor binding proteins; (vi) coagulation and coagulation-related proteins, such as, among others, factor VIII, tissue factor, von Willebrand factor, protein C, alpha-1-antitrypsin, plasminogen activators, such as urokinase and tissue plasminogen activator (“t-PA”), bombazine, thrombin, and thrombopoietin; (vii) other blood and serum proteins, including but not limited to albumin, IgE, and blood group antigens; (viii) colony stimulating factors and receptors thereof, including the following, among others, M-CSF, GM-CSF, and G-CSF, and receptors thereof, such as CSF-1 receptor (c-fms); (ix) receptors and receptor-associated proteins, including, for example, flk2/flt3 receptor, obesity (OB) receptor, LDL receptor, growth hormone receptors, thrombopoietin receptors (“TPO-R,” “c-mpl”), glucagon receptors, interleukin receptors, interferon receptors, T-cell receptors, stem cell factor receptors, such as c-Kit, and other receptors; (x) receptor ligands, including, for example, OX40L, the ligand for the OX40 receptor; (xi) neurotrophic factors, including bone-derived neurotrophic factor (BDNF) and neurotrophin-3, -4, -5, or -6 (NT-3, NT-4, NT-5, or NT-6); (xii) relaxin A-chain, relaxin B-chain, and prorelaxin; (xiii) interferons and interferon receptors, including for example, interferon-α, -ρ, and -γ, and their receptors; (xiv) interleukins and interleukin receptors, including IL-1 to IL-33 and IL-1 to IL-33 receptors, such as the IL-8 receptor, among others; (xv) viral antigens, including an AIDS envelope viral antigen; (xvi) other proteins such as, e.g., lipoproteins, calcitonin, glucagon, atrial natriuretic factor, lung surfactant, tumor necrosis factor-alpha and -beta, enkephalinase, Programmed Cell Death 1 (PD-1), Programmed Cell Death Ligand 1 (PD-Li), T cell immunoreceptor with Ig and ITIM domains (TIGIT), RANTES (regulated on activation normally T-cell expressed and secreted), mouse gonadotropin-associated peptide, DNAse, inhibin, activin, integrin, protein A or D, rheumatoid factors, immunotoxins, bone morphogenetic protein (BMP), superoxide dismutase, surface membrane proteins, decay accelerating factor (DAF), HIV envelope, transport proteins, homing receptors, addressins, regulatory proteins, immunoadhesins, myostatins, TALL proteins, including TALL-I, amyloid proteins, including but not limited to amyloid-beta proteins, thymic stromal lymphopoietins (“TSLP”), RANK ligand (“RANKL” or “OPGL”), c-kit, TNF receptors, including TNF Receptor Type 1, TRAIL-R2, angiopoietins, delta-like ligand 3 (DLL3), CD112R, prostate specific membrane antigen (PSMA), and biologically active fragments or analogs or variants of any of the foregoing.

Examples of therapeutic antibodies suitable for the systems and methods described herein include infliximab, bevacizumab, cetuximab, ranibizumab, palivizumab, abagovomab, abciximab, actoxumab, adalimumab, afelimomab, afutuzumab, alacizumab, alacizumab pegol, ald518, alemtuzumab, alirocumab, altumomab, amatuximab, anatumomab mafenatox, anrukinzumab, apolizumab, arcitumomab, aselizumab, altinumab, atlizumab, atorolimiumab, tocilizumab, bapineuzumab, basiliximab, bavituximab, bectumomab, belimumab, bemarituzumab, benralizumab, bertilimumab, besilesomab, bevacizumab, bezlotoxumab, biciromab, bivatuzumab, bivatuzumab mertansine, blinatumomab, blosozumab, brentuximab vedotin, briakinumab, brodalumab, canakinumab, cantuzumab mertansine, cantuzumab mertansine, caplacizumab, capromab pendetide, carlumab, catumaxomab, cc49, cedelizumab, certolizumab pegol, cetuximab, citatuzumab bogatox, cixutumumab, clazakizumab, clenoliximab, clivatuzumab tetraxetan, conatumumab, crenezumab, cr6261, dacetuzumab, daclizumab, dalotuzumab, daratumumab, demcizumab, denosumab, detumomab, dorlimomab aritox, drozitumab, duligotumab, dupilumab, ecromeximab, eculizumab, edobacomab, edrecolomab, efalizumab, efungumab, elotuzumab, elsilimomab, enavatuzumab, enlimomab pegol, enokizumab, enoticumab, ensituximab, epitumomab cituxetan, epratuzumab, erenumab, erlizumab, ertumaxomab, etaracizumab, etrolizumab, evolocumab, exbivirumab, fanolesomab, faralimomab, farletuzumab, fasinumab, fbta05, felvizumab, fezakinumab, ficlatuzumab, figitumumab, flanvotumab, fontolizumab, foralumab, foravirumab, fresolimumab, fulranumab, futuximab, galiximab, ganitumab, gantenerumab, gavilimomab, gemtuzumab ozogamicin, gevokizumab, girentuximab, glembatumumab vedotin, golimumab, gomiliximab, gs6624, ibalizumab, ibritumomab tiuxetan, icrucumab, igovomab, imciromab, imgatuzumab, inclacumab, indatuximab ravtansine, infliximab, intetumumab, inolimomab, inotuzumab ozogamicin, ipilimumab, iratumumab, itolizumab, ixekizumab, keliximab, labetuzumab, lebrikizumab, lemalesomab, lerdelimumab, lexatumumab, libivirumab, ligelizumab, lintuzumab, lirilumab, lorvotuzumab mertansine, lucatumumab, lumiliximab, mapatumumab, maslimomab, mavrilimumab, matuzumab, mepolizumab, metelimumab, milatuzumab, minretumomab, mitumomab, mogamulizumab, morolimumab, motavizumab, moxetumomab pasudotox, muromonab-cd3, nacolomab tafenatox, namilumab, naptumomab estafenatox, narnatumab, natalizumab, nebacumab, necitumumab, nerelimomab, nesvacumab, nimotuzumab, nivolumab, nofetumomab merpentan, ocaratuzumab, ocrelizumab, odulimomab, ofatumumab, olaratumab, olokizumab, omalizumab, onartuzumab, oportuzumab monatox, oregovomab, orticumab, otelixizumab, oxelumab, ozanezumab, ozoralizumab, pagibaximab, palivizumab, panitumumab, panobacumab, parsatuzumab, pascolizumab, pateclizumab, patritumab, pemtumomab, perakizumab, pertuzumab, pexelizumab, pidilizumab, pintumomab, placulumab, ponezumab, priliximab, pritumumab, PRO 140, quilizumab, racotumomab, radretumab, rafivirumab, ramucirumab, ranibizumab, raxibacumab, regavirumab, reslizumab, rilotumumab, rituximab, robatumumab, roledumab, romosozumab, rontalizumab, rovelizumab, ruplizumab, samalizumab, sarilumab, satumomab pendetide, secukinumab, sevirumab, sibrotuzumab, sifalimumab, siltuximab, simtuzumab, siplizumab, sirukumab, solanezumab, solitomab, sonepcizumab, sontuzumab, stamulumab, sulesomab, suvizumab, tabalumab, tacatuzumab tetraxetan, tadocizumab, talizumab, tanezumab, taplitumomab paptox, tarlatamab, tefibazumab, telimomab aritox, tenatumomab, tefibazumab, teneliximab, teplizumab, teprotumumab, tezepelumab, TGN1412, tremelimumab, ticilimumab, tildrakizumab, tigatuzumab, TNX-650, tocilizumab, toralizumab, tositumomab, tralokinumab, trastuzumab, TRBS07, tregalizumab, tucotuzumab celmoleukin, tuvirumab, ublituximab, urelumab, urtoxazumab, ustekinumab, vapaliximab, vatelizumab, vedolizumab, veltuzumab, vepalimomab, vesencumab, visilizumab, volociximab, vorsetuzumab mafodotin, votumumab, zalutumumab, zanolimumab, zatuximab, ziralimumab, zolimomab aritox, or variants of any of the foregoing.

In accordance with the systems and methods described herein, a sample may comprise two or more of any of the foregoing therapeutic antibodies, or antigen-binding fragments thereof. Exemplary combinations of antigen-binding proteins that may be co-formulated in a sample for analysis as described herein include, but are not limited to, coformulations of antibodies that binds to specifically to PD-1 and at least one additional antibody described herein. In some embodiments, the sample may comprise an anti-PD-1 antibody and an antigen-binding protein (e.g., a BiTE® molecule) that specifically binds to DLL3 (e.g., tarlatamab), PSMA, CD112R, and/or TIGIT. For example, the sample may comprise an anti-PD-1 antibody co-formulated with an antibody that specifically binds to TIGIT and an antibody that specifically binds to CD112R. In other embodiments, the sample may comprise an anti-PD-1 antibody co-formulated with a BiTE® molecule that specifically binds to DLL3 or a BiTE® molecule that specifically binds to PSMA. The present disclosure is not limited to these particular combinations of antigen-binding proteins, however.

### System Reagents

The disclosed system comprises donor beads and acceptor beads, that, when brought in proximity to each other via a biological interaction, a cascade of chemical reactions acts to produce a detectable signal (e.g., a chemiluminescent signal). The terms “bead” and “particle” are used herein interchangeably and refer to a solid support that is typically substantially spherical, though other morphologies may also be suitable for some systems. In exemplary embodiments, donor beads and acceptor beads may be latex-based and about 200 nm-300 nm in diameter (e.g., about 250 nm in diameter). In some embodiments, donor and acceptor beads may be coated with a hydrogel to minimize non-specific binding and self-aggregation, and provide reactive aldehyde groups for conjugating biomolecules to the bead surface. A bead comprising one or more additional moieties will be understood to encompass beads with the additional moiety or moieties immobilized thereon. For example, a bead comprising a photosensitizer and a binding pair member will be understood to encompass a bead conjugated to a photosensitizer and conjugated to a binding pair member.

The terms “solid phase” and “solid support” are used interchangeably herein and refer to any material that can be used to attach and/or attract and immobilize one or more binding members. The term “immobilized,” as used herein, refers to a stable association of a binding member with a surface of a solid support. The terms “binding pair member” and “binding member” are used interchangeably herein and refer to one of two or more different molecules that specifically recognize the other molecule compared to substantially less recognition of other molecules. By way of example, when an antibody or other entity (e.g., antigen binding protein) “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. Accordingly, the two binding pair members will bind to each other more tightly than to other molecules.

The energy transfer from donor bead to acceptor bead is mediated, in part, by the interaction of members of a binding pair that are brought together via a biological interaction. Thus, in exemplary embodiments, the first binding pair member is configured to specifically bind to the second binding pair member. Suitable binding pairs that may be utilized in the system described herein include, but are not limited to, streptavidin-biotin, affinity tag peptide and anti-affinity tag antibody (such as polyhistidine tag and anti-polyhistidine antibody, for example hexahistidine (His6)-anti-His6 antibody, or FLAG peptide-anti-FLAG antibody), and GST-glutathione, as well as specific antibodies targeting post-translational modifications, such as various histone methylation loci. In some embodiments, the first binding pair member comprises streptavidin and the second binding pair member comprises biotin. It will be appreciated that streptavidin is produced from the bacterium Streptomyces avidinii, and streptavidin homo-tetramers have an extraordinarily high affinity for biotin (also known as vitamin B7 or vitamin H). The streptavidin-biotin binding pair is used extensively in molecular biology and bionanotechnology due to its resistance to organic solvents, denaturants (e.g. guanidinium chloride), detergents (e.g. SDS, Triton X-100), proteolytic enzymes, and temperature and pH extremes. Without being limited by theory, biotin has been shown to bind preferentially to free lysine residues. Accordingly, nearly any polypeptide, especially any polypeptide comprising lysine, is contemplated to be capable of biotinylation. In other exemplary embodiments, the first binding pair member comprises hexahistidine (His6) and the second binding pair member comprises an anti-His6 antibody; the first binding pair member comprises a FLAG peptide and the second binding pair member comprises an anti-FLAG antibody; or the first binding pair member comprises glutathione-S-transferase (GST) and the second binding pair member comprises glutathione.

In some embodiments, each donor bead may comprise (e.g., be conjugated to) a photosensitizer and a first binding pair member. In this manner, for example, the photosensitizer in the “donor” bead converts ambient oxygen to a more excited singlet state upon light excitation. The term “photosensitizer,” as used herein, refers to a molecule which absorbs light, generating a reactive singlet oxygen, and transfers the energy from the incident light to another nearby molecule. Examples of photosensitizers include porphyrin, phthalocyanines, and bacteriochlorin derivatives. In some embodiments, the photosensitizer comprises a phthalocyanine, which can excite ambient oxygen into a singlet state following high energy irradiation at about 680 nm.

The disclosed system also comprises acceptor beads, which typically contain a detectable label that reacts with the singlet state oxygen produced from the donor bead. The term “detectable label,” as used herein, refers to a moiety that can produce a signal that is detectable by visual or instrumental means. The detectable label may be, for example, a signal-producing substance, such as a chromagen, a fluorescent compound, an enzyme, a chemiluminescent compound, a radioactive compound, etc. In some embodiments, the acceptor beads are conjugated to a chemiluminescent dye. Generally, the acceptor beads are embedded with at least two different chemical dyes, such as, for example, a thioxene-based dye, anthracene, and rubrene (TAR). In some embodiments, however, the acceptor beads contain at least a thioxene-based dye and either europium (Eu), terbium (Tb), or samarium (Sm) (see, e.g., Eglen et al, supra). In such embodiments, the singlet oxygen initially reacts with thioxene to generate light that is transferred to Eu, Tb, or Sm, which subsequently emit light at about 520-645 nm.

In addition to a chemiluminescent dye (e.g., Eu, Tb, Sm), each acceptor bead may comprise (e.g., be conjugated to) an anti-idiotype antibody that binds to either of the two or more different antigen-binding proteins. The term “idiotype,” as used herein, refers to a set of one or more antigenic determinants (“idiotopes”) that are specific to the variable region of an antibody molecule. Idiotypes are highly conformational epitopes that generally span CDR regions, but they can include non-CDR sequences of the VH and VL domains. Idiotypes may be subdivided into those that reside at the antigen-binding site of the antibody molecule (i.e., the “paratope”) and those on the areas adjacent to this site (i.e., framework determinants). The term “anti-idiotype” antibody, as used herein, refers an antibody directed against an idiotypic determinant.

It will be appreciated that an anti-idiotype antibody binds specifically to an idiotype of an antigen binding protein, which refers to the anti-idiotype antibody binding more tightly to the idiotype of an antigen binding protein than to other molecules. Depending on the idiotypic determinant, anti-idiotype antibodies can be classified as “Ab2 alpha,” “Ab2 beta,” “Ab2 gamma,” or “Ab3.” Ab2 alpha antibodies are directed against idiotypes which are distinct from the antigen-binding site (paratope) on Ab1. Ab2 alpha anti-idiotypic antibodies recognize Ab1 framework region antigens, and are also referred to as “antigen-noninhibitable” since the idiotype/anti-idiotype interaction cannot be inhibited by a hapten that binds specifically to the antigen-binding site. Ab2 beta anti-idiotypic antibodies bind the antigen-binding site of the antibody molecule and compete with a target antigen. Thus, Ab2 beta antibodies are also referred to as “antigen-inhibitable.” Ab2 gamma anti-idiotype antibodies are directed against idiotypes located close to, rather than within, the antigen-binding site. Ab2 gamma antibodies recognize paratope-associated idiotypes and are antigen-inhibitable, as their binding causes alteration of 3-D structure of paratope within the antigen binding site. Anti-idiotypic antibodies are commonly used in art for preclinical pharmacokinetic (PK) analysis of antibody drug products. Idiotypes and anti-idiotype antibodies are described in detail in, for example, Mahmoud Abu-Shakra, Yehuda Shoenfeld,10—Idiotypes And Anti-Idiotypes, Editor(s): Yehuda Shoenfeld, M. Eric Gershwin, Pier Luigi Meroni, Autoantibodies (Second Edition), Elsevier, 2007, pp 69-76. Methods for screening and selecting an anti-idiotype antibody with specificity for a particularly variable region are described in, e.g., Salimi-Moosavi et al., Analytical Biochemistry, 470: pages 52-60 (2015).

In some embodiments, each acceptor bead may comprise (e.g., be conjugated to) a molecule other than an anti-idiotype antibody that binds to either of the two or more different antigen-binding proteins. For example, an acceptor bead may comprise a target antigen to which at least one of the antigen-binding proteins in the sample specifically binds. In such embodiments, the target antigen desirably is in a format that is compatible for binding to the acceptor bead and does not cross-react with other antigen-biding proteins (e.g., therapeutic antibodies) in the coformulated sample.

In some embodiments, the disclosed system comprises a first acceptor bead comprising (e.g., conjugated to) a first anti-idiotypic antibody that binds to one of the two or more antigen-binding proteins in the sample and a second different acceptor bead comprising (e.g., conjugated to) a second anti-idiotypic antibody that binds to the other of the two or more antigen-binding proteins in the sample. Moreover, it will be appreciated that, in such “multiplex” systems, first, second, and/or subsequent acceptor beads are conjugated to chemiluminescent dyes that emit signals of different wavelengths for ease of visualization. For example, the system may comprise (i) a first acceptor bead comprising (e.g., conjugated to) a first chemiluminescent dye (e.g., terbium) and a first anti-idiotype antibody that binds to a first antigen-binding protein in the sample and (ii) a second acceptor bead comprising (e.g., conjugated to) a second chemiluminescent dye (e.g., europium) and a second anti-idiotype antibody that binds to a second antigen-binding protein in the sample. In embodiments where the sample comprises more than two (e.g., 3, 4, 5, or more) different antigen-binding proteins, acceptor beads comprising (e.g., conjugated to) anti-idiotypic antibodies that bind to each of the 3, 4, 5, or more different antigen-binding proteins desirably are included in the system.

In some embodiments, the system further comprises conjugate proteins, wherein each conjugate protein comprises either of the two or more different antigen-binding proteins and a second binding pair member. In other words, a conjugate protein in the context of the present disclosure comprises a different form of the antigen-binding proteins in the sample. For example, the antigen-binding protein of the conjugate protein may be conjugated to the binding pair member. As used herein, the term “conjugate” refers to a molecule comprised of at least two components that are joined together to form a larger biomolecule. For example, a conjugate may include the fusion of two proteins (a “protein conjugate”) or the fusion of a small molecule with a protein or peptide. Protein conjugation (also known as “crosslinking”) may be accomplished by any suitable method known in the art for chemically (e.g., covalently) joining two different peptides or polypeptides. In some embodiments, for example, crosslinking agents that contain two or more reactive ends capable of chemically attaching to specific functional groups (primary amines, sulfhydryls, etc.) on proteins or other molecules may be used (see, e.g., Mattson, G., et al., Molecular Biology Reports, 17: 167-183 (1993)).

By way of example, a sample of interest may comprise two different therapeutic antibodies (“Ab1” and “Ab2”). In such embodiments, the system may comprise a first conjugate protein comprising Ab1 conjugated to a second binding pair member as described herein (e.g., biotin) and a second conjugate protein comprising Ab2 also conjugated to the second binding pair member (e.g., biotin). Thus, in some embodiments, the disclosed system may comprise a first conjugate protein comprising a first antigen-binding protein conjugated to biotin and a second conjugate protein comprising the second antigen-binding protein conjugated to biotin.

### Samples and Kits

The term “sample” and variations of this root term has its ordinary and customary meaning as would be understood by a person of ordinary skill in view of this disclosure. It refers to a composition that may contain two or more antigen-binding proteins as described herein, such as in vitro or synthetic samples obtained from the manufacturing of an antigen-binding protein. For example, a sample may be a composition or formulation comprising the two or more antigen-binding proteins and at least one pharmaceutically acceptable carrier (also referred to herein as a “pharmaceutical composition”). For example, a sample may be an in-process sample from the manufacture of a pharmaceutical composition.

Acceptable composition or formulation materials for antigen-binding proteins as described herein (e.g., therapeutic antibodies) preferably are nontoxic to recipients at the dosages and concentrations employed. In certain embodiments, a pharmaceutical composition may contain formulation materials for modifying, maintaining or preserving, for example, the pH, osmolality, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, sucrose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, e.g., Remington's Pharmaceutical Sciences, 18^{th} Edition, (A. R. Genrmo, ed.), 1990, Mack Publishing Company.

A suitable vehicle or carrier for the composition may be water for injection, physiological saline solution or artificial cerebrospinal fluid, possibly supplemented with other materials common in compositions for parenteral administration. Neutral buffered saline or saline mixed with serum albumin are further exemplary vehicles.

In certain embodiments, buffers are used to maintain the composition at physiological pH or at a slightly lower pH, typically within a pH range of from about 5 to about 8, such as about 6 to about 8. For example, the pH of the composition may be about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0.

The components of the system described herein may be provided in the form of a kit, i.e., a packaged combination of reagents in predetermined amounts with instructions for use. In exemplary aspects, the kit may comprise the sample, donor beads, acceptor beads, and protein conjugates, all of which may be packaged in one or more containers. In some embodiments, the kit further comprises a substrate. The substrate may be configured for immobilization of the donor beads, acceptor beads, and/or conjugate proteins on the substrate, or the donor beads, acceptor beads and/or conjugate proteins of the kit may be immobilized on the substrate. Examples of suitable substrates include tubes, beads, microparticles, nanoparticles, microplates, multi-well plates, gels, colloids, sheets, and chips.

### Assay Methods

The disclosure further provides methods for analyzing compositions containing two or more antigen-binding proteins (e.g., therapeutic antibodies). The methods provided herein desirably are homogenous, proximity-based assays. “Homogenous” assays yield read-outs without having to separate the analyte(s) of interest (e.g., antigen-binding proteins) from the biomolecules used to detect them. In contrast, “heterogeneous” assays require one or more steps of separation, where unbound analyte (e.g., antigen-binding proteins) and/or unbound detection molecules must be washed away. Proximity-based assays, for example no-wash assays such as ALPHASCREEN® methodologies (e.g., ALPHASCREEN®, ALPHALISA®, and ALPHAPLEX™, PerkinElmer, Inc., Waltham, MA), typically involve bead-based chemistry to study interactions between molecules in a microplate. ALPHASCREEN® technology has been routinely utilized in high throughput screening assays to quantify analyte accumulation or depletion, bimolecular interactions, and post-translational modifications (see, e.g., Ullman et al., Proc. Natl. Acad. Sci. USA, 91: 5426-5430 (1994); and Yasgar et al., Methods Mol Biol., 1439: 77-98 (2016) doi:10.1007/978-1-4939-3673-1_5).

In some embodiments, provided herein is method of determining a presence, absence, or concentration of two or more different antigen-binding proteins in a sample, which method comprises (1) incubating the sample with the system described herein; (2) exposing the incubated sample to light excitation; and (3) detecting at least one chemiluminescent signal following light excitation and comparing the detected chemiluminescent signal to a predetermined reference value. Descriptions of the donor beads, acceptor beads, protein conjugates, antigen-binding proteins, and samples set forth herein in connection with the aforementioned system also are applicable to those same aspects of the disclosed methods.

The sample may be contacted and incubated with the various components of the system in any suitable order for any suitable duration. For example, the sample may be contacted with donor beads, acceptor beads, and conjugate proteins sequentially or simultaneously. In some embodiments, the sample is applied to a substrate (e.g., wells of microplate), followed by sequential introduction of conjugate proteins, acceptor beads, and finally, donor beads. In other embodiments, the sample is applied to a substrate, followed by the sequential introduction of acceptor beads, conjugate proteins, and finally donor beads. In some embodiments, any of the donor beads, acceptor beads, and/or conjugate proteins may be immobilized on a substrate, followed by introduction of the sample. For some applications, it may be appropriate to simultaneously introduce the acceptor beads, conjugate proteins, and donor beads to the sample. The choice of an appropriate assay format will be apparent to one of ordinary skill in the art.

The sample and system components may be incubated for any length of time that is sufficient to allow for interaction between donor and acceptor beads. In some embodiments, the sample is incubated with the system for at least 15 minutes (e.g., 20 minutes, 30 minutes, 45 minutes, or 60 minutes), but not greater than 3 hours (e.g., 2.5 hours, 2 hours, 1.5 hours). In some embodiments, incubation times of about 30 minutes to about 2 hours (e.g., 35 minutes, 50 minutes, 75 minutes, or 100 minutes) are preferred. An advantage of the disclosed methods is that they may be completed in a time-efficient manner, which is due in part to very high signal/background ratios. As such, a wide range of conditions are suitable for generating an appropriate signal.

Whatever format is employed, the method involves exposing the sample, once incubated with the system, to light excitation, such that the photosensitizer in donor beads converts oxygen (O₂) into an excited singlet state (¹O₂). Excitation may occur at any wavelength of light that is capable of producing singlet oxygen. In some embodiments, the light excitation wavelength may be about 500 nm to about 800 nm (e.g., about 520 nm, 550 nm, 580 nm, 600 nm, 620 nm, 650 nm, 680 nm, 700 nm, 720 nm, 750 nm, or 780 nm). For example, the light excitation wavelength may be about 600 nm to about 700 nm (e.g., about 610 nm, 620 nm, 630 nm, 640 nm, 650 nm, 660 nm, 670 nm, 680 nm, or 690 nm). Desirably, a wavelength of about 680 nm is used for light excitation of the incubated sample.

A chemiluminescent signal is generated within an acceptor bead if it is located in close proximity (e.g., about 200 nm, 150 nm, 100 nm, 50 nm, 25 nm, 5 nm, or less) to a donor bead to receive singlet oxygen. As discussed above, depending on the number of acceptor beads employed, which in turn depends on the number of antigen-binding proteins in a sample, chemiluminescent signals of varying emission wavelengths may be detected. Chemiluminescent dyes (e.g., Tb, Eu, and Sm) that may be used desirably emit a signal at wavelengths between about 500 nm to about 700 nm (e.g., about 520 nm, 550 nm, 580 nm, 600 nm, 620 nm, 650 nm, or 680 nm). In some embodiments, the emission wavelength may be about 520 nm to about 620 nm (e.g., about 530 nm, about 545 nm, about 560 nm, about 575 nm, about 590 nm, or about 615 nm). For example, the terbium emission peak is at 545 nm, the europium emission peak is at 615 nm, and the samarium emission peak is at 645 nm.

Thus, in some embodiments the method further comprises assaying the sample for the presence of at least one chemiluminescent signal following light excitation. As described above, a system encompassed by the present disclosure may comprise (i) a first acceptor bead conjugated to a first chemiluminescent dye and a first anti-idiotype antibody that binds to a first antigen-binding protein in the sample and (ii) a second acceptor bead conjugated to a second chemiluminescent dye and a second anti-idiotype antibody that binds to a second antigen-binding protein in the sample. In such embodiments, for example, the method may comprise detecting a first signal emitted from the first chemiluminescent dye and a second signal emitted from the second chemiluminescent dye. The first signal and second signal may comprise two different wavelengths. For example, the wavelength of each of the first and second signals may be about 520 nm to about 620 nm (e.g., about 530 nm, about 540 nm, about 550 nm, about 560 nm, about 570 nm, about 580 nm, about 590 nm, about 600 nm, or about 610 nm). In some embodiments, the wavelength of the first signal is about 545 nm and the wavelength of the second signal is about 615 nm.

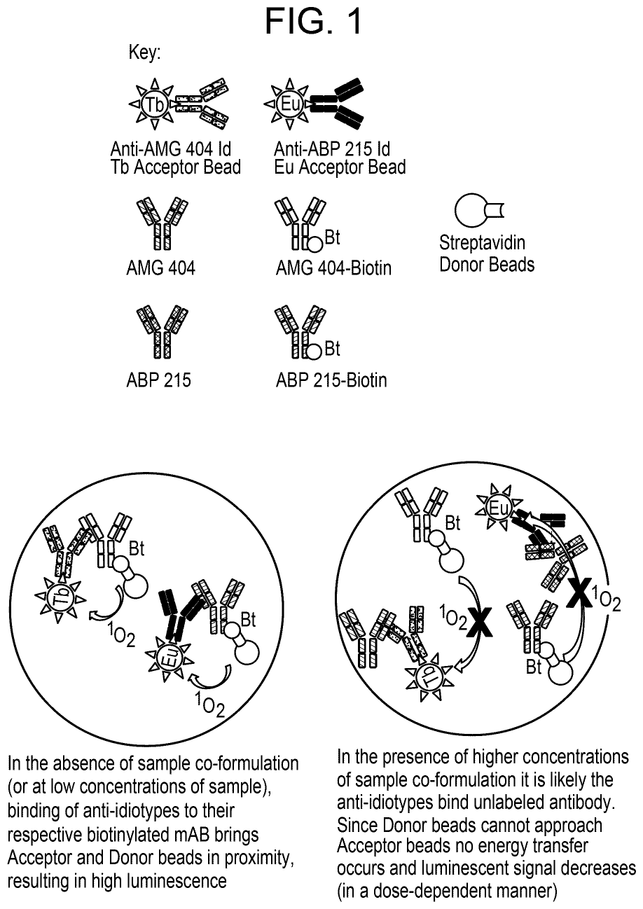

Proximity assays are frequently used in the art to assess the presence of an analyte in a sample via interaction of donor beads with acceptor beads and subsequent transfer of chemical energy from singlet oxygen from the donor bead to the acceptor bead, producing a chemiluminescent signal. In such cases, the intensity of the chemiluminescent signal is directly proportional to the concentration of the analyte in the sample. As used herein, the term “intensity” means the amount or degree of strength of electricity, light, heat, or sound per unit area or volume. In other aspects, proximity assays may be designed such that disruption of a donor-acceptor interaction by an analyte of interest results in depletion of chemiluminescent signal intensity. In such cases, the intensity of the chemiluminescent signal is inversely proportional to the concentration of the analyte in the sample. For example, in some embodiments of the method, detection of one or more chemiluminescent signals indicates that the sample lacks the two or more different antigen-binding proteins or comprises the two or more different antigen-binding proteins at low concentrations, while the absence of a chemiluminescent signal, or a signal of decreased intensity, indicates that the sample comprises high concentrations of the two or more different antigen-binding proteins. An exemplary assay format is schematically illustrated in FIG. 1, which illustrates that, in the absence of co-formulated antibodies (or low concentrations of co-formulated antibodies in a sample), binding of anti-idiotype antibodies on acceptor beads to their respective conjugate protein (e.g., a biotinylated monoclonal antibody) brings acceptor and donor beads in proximity, leading to a high signal intensity. Using a sample comprising high concentrations of co-formulated antibodies, the anti-idiotype antibodies on acceptor beads likely bind to their respective unlabeled antibody target in the sample, and donor beads are unable to approach acceptor beads to transfer energy from singlet oxygen, leading to a decrease in luminescent signal. Such a depletion of signal may occur in a dose-dependent manner.

In accordance with the inventive method, the intensity of the chemiluminescent signal(s) detected in a sample may be compared to a predetermined reference value, such as a predetermined threshold value. The term “predetermined reference value” refers to an assay value, or defined amount of analyte, that is used to assess an assay result. For example, a predetermined reference value may be a chemiluminescent signal corresponding to a known quantity of therapeutic protein. Predetermined reference values may be based on calibration curves as described herein. A reference value may be based upon a concentration specification of each therapeutic protein in a pharmaceutical formulation. The terms “predetermined threshold value” and “predetermined cut-off value” are used interchangeably herein to refer to an assay value, or defined amount of an analyte, that is used to assess the concentration of an analyte in an unknown sample by comparing the assay results against the predetermined threshold or cut-off value. While the present disclosure may provide exemplary predetermined threshold levels, it is well-known that cut-off values may vary depending on the nature of the assay (e.g., antibodies employed, etc.). It further is well within the ordinary skill in the art to adapt the disclosure herein for other samples and antigen-binding proteins to obtain assay-specific reference or cutoff values for such assays based on this disclosure.

In some embodiments, the predetermined reference value may be a chemiluminescent signal corresponding to an antigen-binding protein concentration of about 0.05 μg/mL to about 150 μg/mL. Thus, in some embodiments, a predetermined reference value may correspond to an antigen binding concentration of about 1 μg/mL, about 5 μg/mL, about 10 μg/mL, about 20 μg/mL, about 30 μg/mL, about 40 μg/mL, about 50 μg/mL, about 60 μg/mL, about 70 μg/mL, about 80 μg/mL, about 90 μg/mL, about 100 μg/mL, about 110 μg/mL, about 120 μg/mL, about 130 μg/mL, about 140 μg/mL, or a range defined by any two of the foregoing values, for example about 20 μg/mL to about 140 μg/mL.

In other embodiments, the intensity of the chemiluminescent signal(s) detected in a sample may be compared to a predetermined reference curve for each antigen-binding protein in the sample. For example, a predetermined reference curve may be a dose-response curve that is established for each antigen-binding protein in a co-formulation sample to determine the acceptable binding range for each antigen-binding protein. Methods for generating dose-response curves in the context of the present disclosure are described in detail in the Examples.

In some embodiments, the methods described herein further comprise quantifying the two or more antigen-binding proteins in the sample. In embodiments where chemiluminescent signal intensity is detected, spectrophotometric analysis may be used to quantify antigen-binding protein concentration. As used herein, the terms “spectrophotometric” or “photometric” analysis refer to the determination of materials in a substance or sample by measuring the amount of light they absorb in the infrared, visible, or ultraviolet region of the spectrum. In spectrophotometric analysis, the course of a reaction is followed by measuring a change in how much light the assay solution (e.g., sample) absorbs. Thus, in some embodiments, the concentrations of each of the antigen-binding proteins in the sample may be quantified by measuring the UV absorbance at 280 nm. It will be appreciated that at 280 nm wavelength, the aromatic amino acids tryptophan (Trp) and tyrosine (Tyr) exhibit strong light absorption, and to a lesser extent cysteine groups forming disulfide bonds (Cys-Cys) also absorb. Consequently, absorption of proteins and peptides at 280 nm is proportional to the content of these amino acids.

In certain embodiments, the concentration of each of the antigen-binding proteins in the sample may be determined by generating a calibration curve (also commonly referred to as standard curve or working curve). A “calibration curve” is a general method for determining the concentration of a substance in an unknown sample by comparing the unknown to a set of standard samples of known concentration. The responses of the standards are used to plot or calculate a standard curve. Absorbance values of unknown samples are then interpolated onto the plot or formula for the standard curve to determine their concentrations. When the calibration curve has an adequate linearity over a wider range in the region of quantitative analysis, the calibration curve can be prepared with a relatively smaller number of standard samples, which are near the upper limit, lower limit, and in the intermediate point in the determination range of the quantitative analysis.

In a sample comprising two or more antigen-binding proteins, as described herein, at least two calibration curves may be generated and utilized to quantify the amount of each antigen-binding protein detected in the disclosed methods. For example, a first calibration curve may be generated at a first wavelength that is emitted by a first acceptor bead, while a second calibration curve may be generated at a second wavelength that is emitted by a second acceptor bead.

In some aspects, the concentration of an antigen-binding protein in a sample may be characterized in terms of a half maximal inhibitory concentration (IC₅₀), which is a measure of the effectiveness of the antigen-binding protein in inhibiting an interaction between donor and acceptor beads. In other aspects, the concentration of an antigen-binding protein in a sample may be characterized in terms of half maximal effector concentration (EC₅₀), which is a measure of the effectiveness of the antigen-binding in effecting an interaction between donor and acceptor beads.

Other orthogonal methods for determining protein concentration may be employed in order to cross-check or confirm the results obtained by the disclosed methods. Such methods include, but are not limited to, turbidimetric assays, nephelometric assays, and colorimetric assays. In turbidimetric and nephelometric assays, a protein is quantified from the change in the turbidity of the reaction mixture based on the agglutination of the protein and a protein-specific binding partner. In colorimetric assays, a protein may be quantified with the aid of a color reagent. Colorimetric assays are characterized by formation, change, or depletion of color in the presence of the protein to be quantified. Exemplary colorimetric assays include the Coomassie blue G-250 dye-binding (Bradford), bicinchoninic acid (BCA), and Lowry assay.

The following example further illustrates the invention but, of course, should not be construed as in any way limiting its scope.

### Example

This example describes the generation of a system for detecting and quantifying two different antigen-binding proteins in a sample and use thereof.

Reagents specific for two antibody targets, denoted “Ab1” and “Ab2” were generated. In particular, unlabeled Ab1 and Ab2 were biotinylated at Amgen and are referred to as Ab1-Biotin and Ab2-Biotin, respectively. In cases where glycerol or amines were present in the unlabeled antibody formulation buffer, the unlabeled antibody was desalted prior to biotinylation using a ZEBA™ spin desalting column (7K MWCO) and exchanged into 1× Dulbecco's Phosphate Buffered Saline (D-PBS, pH 7.0) per standard protocol. Biotinylation was performed by mixing unlabeled antibody in 100 mM Borax (pH 9.3) buffer with the EZ-LINK™ Sulfo-NHS-LC-LC-Biotin reagent, in which covalent linkages are formed between NHS-esters of the biotin reagent and primary amines on the antibody surface. The molar ratio of biotin to unlabeled antibody (referred to as the “challenge ratio”) may be 20:1, 10:1, 5:1, 2:1, 1:1, 1:2, 1:5, 1:10, or 1:20. Samples were mixed constantly (30-60 minutes, 25° C.), and then purified on a ZEBA™ spin desalting column (7K MWCO) and exchanged into 1× Dulbecco's Phosphate Buffered Saline (D-PBS, pH 7.0) per standard protocol. Final concentrations of biotinylated antibody (Ab1-Biotin or Ab2-Biotin) were determined via UV-VIS spectrophotometry using a wavelength of 280 nm. The degree of biotinylation was determined using the Pierce™ Biotin Quantitation Kit per the manufacturer's protocol with triplicate UV-VIS spectrophotometric measurements taken at 500 nm. In practice the degree of biotinylation is reported as real numbers to two decimal places, reflecting an average number of biotin molecules per IgG molecule. In this work Ab1-Biotin was determined to have 6.18 biotin molecules per Ab1 molecule, and Ab2-Biotin was determined to have 5.62 biotin molecules per Ab2 molecule. In some embodiments, the degree of biotinylation may be any real number up to but not exceeding the challenge ratio used. For example, for a challenge ratio of 20:1 (molar ratio of biotin to IgG molecules) the degree of biotinylation may be any real number ratio ranging from 20:1 to 0:1, the latter being cases where biotinylation fails; however, in cases of poor biotinylation reaction efficiency it is not impossible for there to be a degree of labeling in which the ratio of biotin is greater than 0 but less than 1 molecule per IgG molecule.

Antibody specific anti-idiotypes for Ab1 and Ab2 were conjugated to acceptor beads containing fluorophores for detection. Initially, Ab1-specific anti-idiotype antibody was directly conjugated to ALPHAPLEX™ 545 (Terbium) Acceptor Beads via a reductive amination of aldehyde groups on the bead surface. Analogously, Ab2-specific anti-idiotype antibody was directly conjugated to ALPHALISA® (Europium) Acceptor Beads. The protocol followed is available from PerkinElmer as a Technical Brief, “Antibody Conjugation to ALPHALISA® Acceptor Beads.” Briefly, acceptor beads were washed by adding 50 μL of 1× PBS (pH 7.4) to a 1 milligram tube of acceptor beads direct from the supplier (PerkinElmer®). Beads were collected by centrifugation (16,000×g, 15 minutes) and supernatant removed. Ab-specific (Abl or Ab2) anti-idiotype antibody were added to a buffer mixture composed of 100 mM HEPES, 20 mM sodium cyanoborohydride, 0.06% Tween-20, pH 7.4. The Ab-specific anti-idiotype antibody mixture was used to resuspend the acceptor bead pellet, and the suspension transferred to a 0.5 mL screw cap tube. Conjugation reaction tubes were incubated overnight (37° C., 18-24 hours) in a tube rotator. Carboxymethoxylamine (CMO) was added to a concentration of roughly 3 mg/mL to block unreacted aldehyde groups, and the reaction tubes were incubated at 37° C. for 1 hour. Ab-specific anti-idiotype antibody-conjugated acceptor beads were collected in a refrigerated centrifuge (16,000×g, 15 minutes, 4° C.), supernatant was removed, and the pellet was washed in 100 mM Tris-HCl (pH 8.0) followed by collection using the previous refrigerated centrifuge conditions. After removing supernatant, the Ab-specific anti-idiotype antibody-conjugated acceptor beads were resuspended in 1× PBS, 0.05% PROCLIN™300 (pH 7.4) at a concentration of 5 mg/mL and stored at 2-8° C. in amber screw cap tubes protected from light. Ab1-specific anti-idiotype antibody or Ab2-specific anti-idiotype antibody were covalently linked to their respective acceptor bead surfaces via reductive amination occurring between accessible lysine residues on the Ab surface and the reactive aldehyde groups on the acceptor bead surface. In some embodiments, Ab1 may be conjugated to ALPHAPLEX™ Acceptor Beads and Ab2 conjugated to ALPHALISA® Acceptor Beads. In other embodiments, Ab1 may be conjugated to ALPHALISA® Acceptor Beads and Ab2 conjugated to ALPHAPLEX™ Acceptor Beads. The details for reagents used in the conjugation reactions are shown in Table 1.

TABLE 1
Material/reagent suppliers and catalog numbers

| Reagent | Supplier | Catalog Number |
|---|---|---|
| ALPHAPLEX ™ 545 Acceptor Beads | PerkinElmer, Inc | 6782002 |
| ALPHALISA ® Acceptor Beads | PerkinElmer, Inc | 6772002 |
| Sodium cyanoborohydride (NaBH₃CN), 5M | Sigma-Aldrich | 296945 |
| solution | | |
| O-(Carboxymethyl)hydroxylamine | Sigma-Aldrich | C13408 |
| hemichloride (carboxymethoxylamine, CMO) | | |
| PBS, pH 7.4 | Gibco | 10010-023 |
| PROCLIN ™-300 | Sigma-Aldrich | 48912-U |
| 100 mM Tris-HCl, pH 8.0 | Amgen Media Sciences | N/A |
| EZ-LINK ™ Sulfo-NHS-LC-LC-Biotin | Thermo Scientific | 21338 |
| ZEBA ™ Spin Desalting Columns, 7K | Thermo Scientifc | 89889 (*Cat. No. |
| MWCO | | varies by column |
| | | volume) |
| 100 mM Borax, pH 9.3 | Amgen Media Sciences | N/A |
| Pierce Biotin Quantitation Kit | Thermo Scientific | 28005 |
| N,N-Dimethylformamide | Sigma-Millipore | 227056-100ML |

The reagents were tested in separate assays specific for each target in which 8-point serial dilutions (ranging from 100 μg/mL to 10 μg/mL in-well concentration) of unlabeled antibody (Ab1 or Ab2) were mixed with their respective biotinylated antibody (Ab1-Biotin or Ab2-Biotin) [0.15 μg/mL in-well concentration] and their respective (Ab1- or Ab2-specific) anti-idiotype antibody-conjugated acceptor beads [10 μg/mL in-well concentration] in 96-well plates [Corning®, Cat. No. 3642], and incubated (1 hour, 25° C.). Streptavidin-coated donor beads [PerkinElmer®, Cat. No. 6760002] were added to each well [40 μg/mL in-well concentration], and reactions returned to incubation (30 minutes, 25° C.). Replicates of each serial dilution were analyzed on a single assay plate to assess standard deviation and percent coefficient of variation (CV). Plates were analyzed using a PerkinElmer ENVISION® 2104 plate reader with excitation occurring at 680 nm, and emission signal at 545 nm was measured first followed by signal emitted at 615 nm corresponding to the excited Terbium and Europium fluorophores, respectively. Raw signal was analyzed using a 4-parameter logistic curve-fit using SOFTMAX® Pro Software v5.4.1 (Molecular Devices, LLC) to generate dose-dependent response curves.

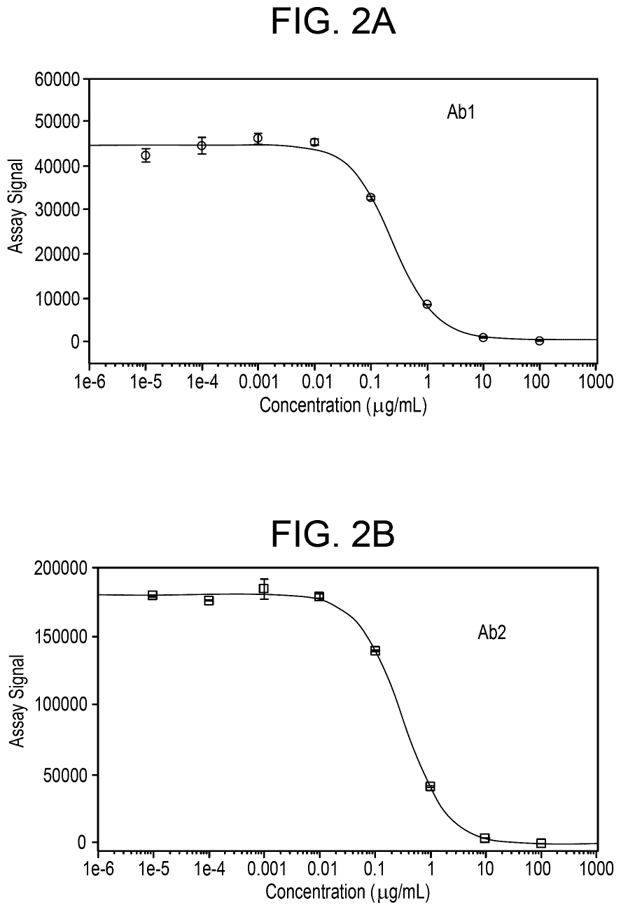

The reagents showed high specificity for their respective reagents, as shown in the dose-dependent response curves of FIGS. 2A and 2B. Low error between replicates was observed.

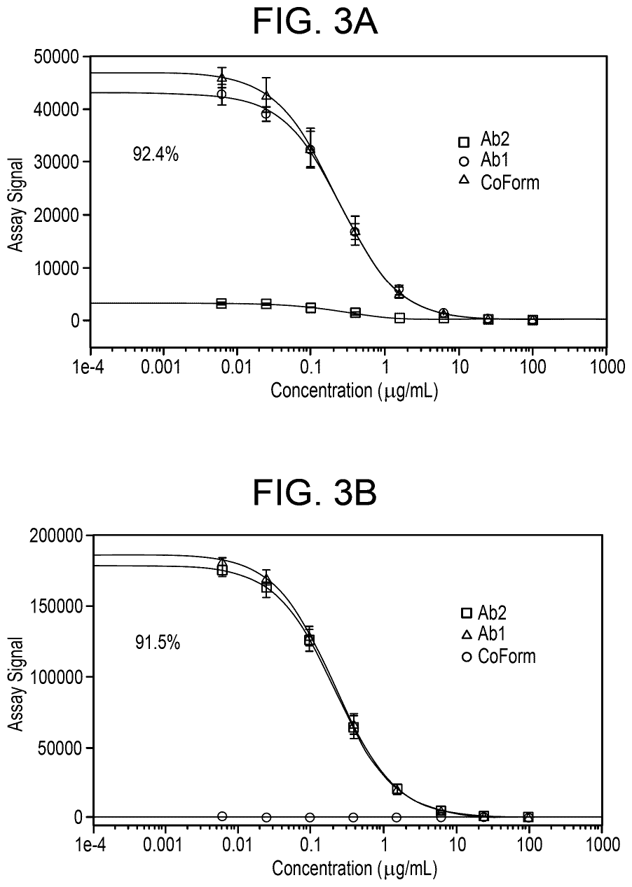

In order to distinguish between the two antibodies in a formulation and establish percent relative binding values, a mock co-formulation experiment with Ab1 and Ab2 was performed. A “CoForm” sample was prepared, containing 10 mg/mL of each antibody in ALPHALISA® Immunoassay Buffer (25 mM HEPES, 0.1% Casein, 1 mg/mL Dextra-500, 0.5% Triton X-100, 0.05% Proclin-300, pH 7.4) (PerkinElmer, Cat. No. ALOOOF). Other reagents included biotinylated Ab1 and Ab2 (Ab1-Biotin and Ab2-Biotin), terbium-labeled Ab1-specific anti-idiotype antibody-conjugated acceptor beads, europium-labeled Ab2-specific anti-idiotype antibody-conjugated acceptor beads, and streptavidin-coated donor beads. In this assay 8-point serial dilutions (ranging from 100 μg/mL to 6.1 ng/mL in-well concentration) of Ab1 only, Ab2 only, and the CoForm sample were analyzed in parallel on a 96-well plate (Corning® 3642) in the presence of the same reagents, except for the biotinylated antibody each received. Specifically, Ab1 serial dilution wells received 0.15 μg/mL (in-well concentration) of only Ab1-Biotin (but no Ab2-Biotin), Ab2 received 0.15 μg/mL (in-well concentration) of only Ab2-Biotin (but no Ab1-Biotin), and the CoForm sample received a mixture containing 0.15 μg/mL each (in-well concentration) of both Ab1-Biotin and Ab2-Biotin. All wells received a mixture of Ab1-specific and Ab2-specific anti-idiotype antibody-conjugated acceptor beads (10 μg/mL each in-well concentration), and plates were then incubated (1 hour, 25° C.). Streptavidin-coated donor beads (PerkinElmer®, Cat. No. 6760002) were added to each well (40 μg/mL in-well concentration), and reactions returned to incubation (30 minutes, 25° C.). Replicates of each serial dilution were analyzed on a single assay plate to assess standard deviation and percent coefficient of variation (CV). For analysis using a 4-parameter logistic curve-fit in SOFTMAX® Pro Software v5.4.1 of the 545 nm (Terbium) channel—corresponding to Ab1 binding—the signal from Ab1 only was used as Reference Standard to determine the percent relative binding of Ab1 in the CoForm sample, and Ab2 only used as Control to show lack of signal (no Ab1-Biotin present to bring the donor beads and anti-Ab1 specific idiotype acceptor beads into proximity) (see FIG. 3A). Similarly, when analyzing the 615 nm (Europium) channel—corresponding to Ab2 binding—Ab2 only signal was used as Reference Standard to determine the percent relative binding of Ab2 in the CoForm sample, and Ab1 only used as Control to show lack of signal (see FIG. 3B). As shown in FIGS. 3A and 3B, the antibodies in co-formulation showed percent relative binding within specification (80-120% binding relative to control) as compared to their respective controls. Further, the results adhered and passed strict internal Assay Acceptance Criteria and Sample Acceptance Criteria.

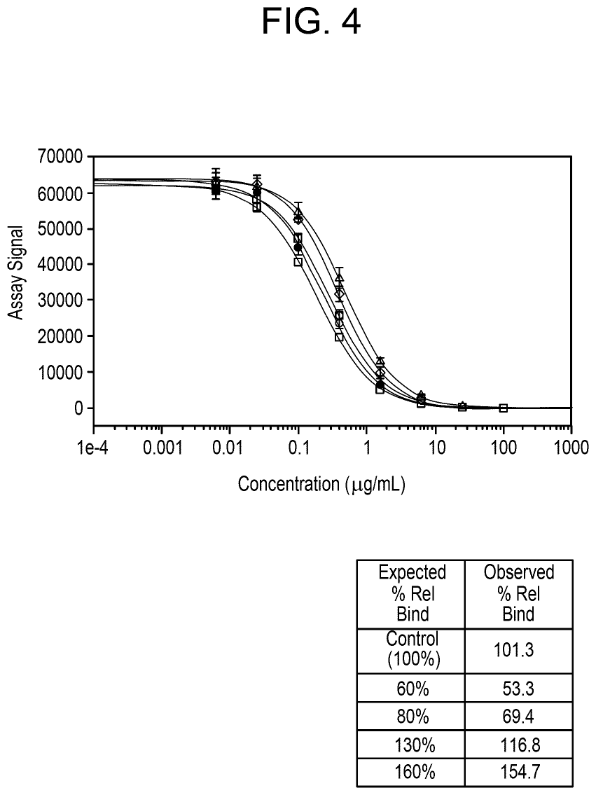

The linearity of the assay was analyzed with respect to Ab1. In a panel of co-formulation samples, a constant concentration (10 mg/mL) of Ab2 was maintained while Ab1 was included at simulated concentrations expected to achieve 60%, 80%, 130%, and 160% percent relative binding. Other reagents included biotinylated Ab1 and Ab2 (Ab1-Biotin and Ab2-Biotin), terbium-labeled Ab1 specific anti-idiotype antibody-conjugated acceptor beads, europium-labeled Ab2 specific anti-idiotype antibody-conjugated acceptor beads, and streptavidin-coated donor beads. In this assay, 8-point serial dilutions (ranging from 100 μg/mL to 6.1 ng/mL in-well concentration) of the simulated co-formulation samples were analyzed in parallel on a 96-well plate (Corning® 3642) in the presence of the same reagents. Specifically, a mixture containing 0.15 μg/mL each (in-well concentration) of both Ab1-Biotin and Ab2-Biotin, and a mixture containing 10 μg/mL each (in-well concentration) of both Ab1 specific anti-idiotype antibody conjugated-acceptor beads (Terbium) and Ab2 specific anti-idiotype antibody conjugated-acceptor beads (Europium) were added to each well of the plate containing simulated co-formulation samples, and plates were then incubated (1 hour, 25° C.). Streptavidin-coated donor beads (PerkinElmer®, Cat. No. 6760002) were added to each well [40 μg/mL in-well concentration], and reactions returned to incubation (30 minutes, 25° C.). Replicates of each serial dilution were analyzed on a single assay plate to assess standard deviation and percent coefficient of variation (CV). Raw signal was analyzed using a 4-parameter logistic curve-fit using SOFTMAX® Pro Software v5.4.1 and percent relative binding of each simulated sample reported. The results of this analysis are shown in FIG. 4. Ab1 exhibited linearity within the normal specification range.

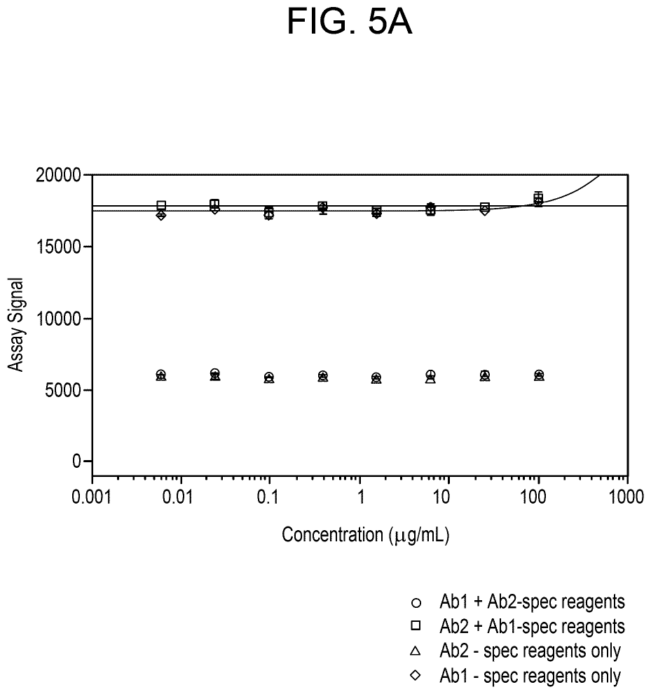

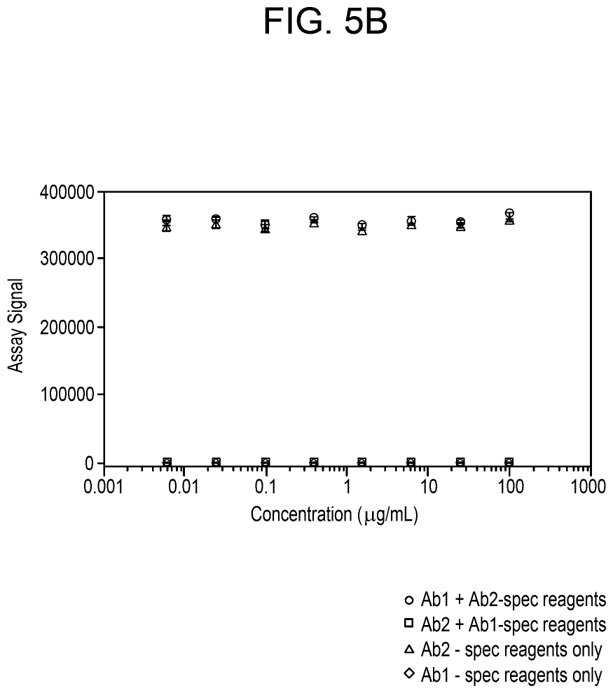

The specificity of the assay was analyzed to ensure there was no cross-reactivity between Ab1 and the Ab2-specific reagents (namely Ab2-specific anti-idiotype antibody and Ab2-Biotin), as well as Ab2 and the Ab1-specific reagents (namely Ab1-specific anti-idiotype antibody and Ab1-Biotin). In this assay, 8-point serial dilutions of Ab1 were mixed with Ab2-Biotin (0.15 μg/mL in-well concentration) and Ab2-specific anti-idiotype antibody-conjugated acceptor beads (10 μg/mL in-well concentration) on a 96-well plate (Corning® 3642). These Ab2-specific reagents were also analyzed on the same assay plate in the absence of Ab1 to show the maximum signal obtained when Ab2-Biotin bound streptavidin-coated donor beads come in proximity of Ab2 specific anti-idiotype antibody-conjugated acceptor beads (via binding between Ab2-Biotin and the anti-Ab2 idiotype). On the same assay plate 8-point serial dilutions of Ab2 were incubated in the presence of Ab1-Biotin(0.15 μg/mL in-well concentration) and Ab1-specific anti-idiotype antibody-conjugated acceptor beads (10 μg/mL in-well concentration). These Ab1-specific reagents were also analyzed on the same assay plate in the absence of Ab2 to show the maximum signal obtained when Ab1-Biotin bound donor beads come in proximity of Ab1 specific anti-idiotype antibody-conjugated acceptor beads. Plates were then incubated (1 hour, 25° C.), followed by addition of streptavidin-coated donor beads (PerkinElmer®, Cat. No. 6760002) to each well (40 μg/mL in-well concentration); plates were returned to incubation (30 minutes, 25° C.). Replicates of each serial dilution, and the no-Ab1 or no-Ab2 controls, were analyzed on the same plate to assess standard deviation and percent coefficient of variation (CV). Raw signal was analyzed using a 4-parameter logistic curve-fit using SOFTMAX® Pro Software v5.4.1. As shown in FIGS. 5A and 5B, the reagents generated for use in this coformulation assay demonstrate high specificity with low potential for cross-reactivity of reagents.

To ensure the choice of acceptor bead conjugation partners was optimal for the assay, the anti-Ab specific idiotypes were conjugated to the opposite acceptor bead as described above. There were no observable differences in raw signal or plotted curves between the initial format and the swapped format.

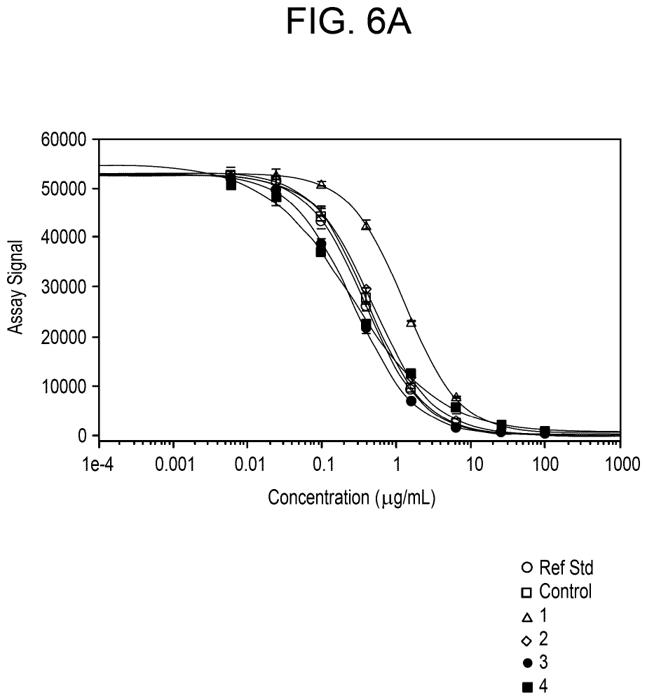

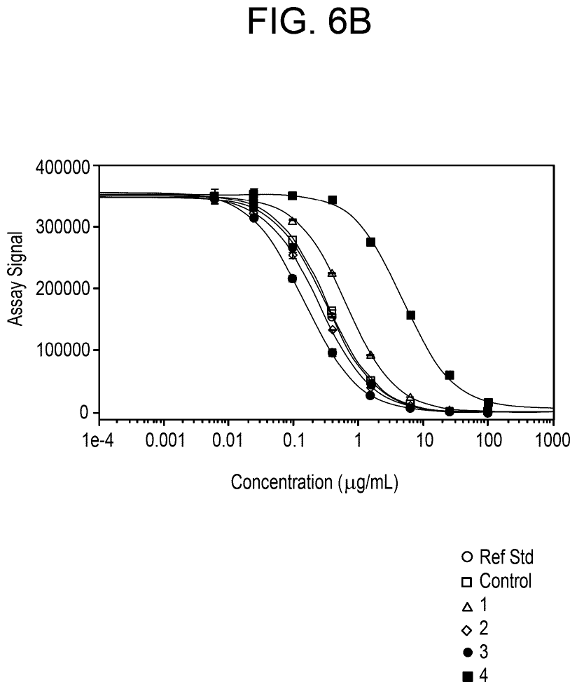

The practical use of the coformulation assay was demonstrated via the analysis of blind mock coformulation samples. The only constraint on the sample preparer was that the concentrations of Ab1 or Ab2 must each be between 0.5-20 mg/mL in each blind mock sample. A CoForm Reference Standard (Ref Std) was prepared having 10 mg/mL each of Ab1 and Ab2 in ALPHALISA® Immunoassay buffer. This mock CoForm Ref Std is used on each assay plate as Reference Standard to measure sample percent relative binding against and in parallel as an Assay Control to ensure the assay and operator are performing as expected (as measured using Assay Acceptance Criteria and Sample Acceptance Criteria). In this assay, 8-point serial dilutions of the blind mock co-formulation samples were analyzed in parallel on a 96-well plate (Corning® 3642) in the presence of the same reagents. Since the samples were blind there were analyzed using the same dilution factor as the Ref Std and Control wells, so all samples were analyzed against 10 mg/mL each of unlabeled Ab1 and Ab2 and their signal would be proportional to the relative amounts of unlabeled Ab1 and Ab2 in the blind mock co-formulation samples. All wells received a mixture containing 0.15 μg/mL each (in-well concentration) of both Ab1-Biotin and Ab2-Biotin, and a mixture containing 10 μg/mL each (in-well concentration) of both Ab1 specific anti-idiotype antibody conjugated-acceptor beads (Terbium) and Ab2 specific anti-idiotype antibody conjugated-acceptor beads (Europium), and plates were then incubated (1 hour, 25° C.). Streptavidin-coated donor beads [PerkinElmer®, Cat. No. 6760002] were added to each well [40 μg/mL in-well concentration], and reactions returned to incubation (30 min., 25° C.). Replicates of each serial dilution were analyzed on a single assay plate to assess standard deviation and percent coefficient of variation (CV). Raw signal was analyzed using a 4-parameter logistic curve-fit using SoftMax® Pro Software v5.4.1 [Molecular Devices] and percent relative binding of each simulated sample reported. Three independent assays (i.e. “determinations”) were performed and the average percent relative binding calculated for Ab1 and Ab2 in each blind mock coformulation sample. FIGS. 6A and 6B show representative graphs for the 545 nm (Terbium) and 615 nm (Europium) signal obtained. The results showed exceptional ability for the assay to determine the concentration of Ab1 or Ab2 in the blind samples in the range of roughly 1-15 mg/mL. At concentrations successively higher or lower than this range the percent error in concentration measurements suggests the assay is unreliable, at least when using the Reference Standard concentration as tested (10 mg/mL each Ab1 and Ab2).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A system for detecting and quantifying two or more different antigen-binding proteins in a sample, which system comprises:
(a) donor beads, wherein each donor bead comprises a photosensitizer and a first binding pair member;
(b) acceptor beads, wherein each acceptor bead comprises a chemiluminescent dye and an anti-idiotype antibody that specifically binds to either of the two or more different antigen-binding proteins and;
(c) conjugate proteins, wherein each conjugate protein comprises either of the two or more different antigen-binding proteins and a second binding pair member,
wherein the first binding pair member is configured to specifically bind to the second binding pair member.

2. The system of claim 2, wherein each of the antigen-binding proteins is an antibody, an antibody fragment, or a bispecific T cell engager (BiTE®) molecule.

3. The system of claim 1, wherein the photosensitizer comprises phthalocyanine.

4. The system of any claim 1, wherein the first binding pair member comprises streptavidin, glutathione S-transferase (GST), or an affinity tag peptide such as a hexahistidine or a FLAG peptide.

5. The system of claim 1, wherein the second binding pair member comprises biotin, glutathione, or anti-affinity tag antibody such as an anti-hexahistidine antibody or an anti-FLAG antibody.

6. The system of claim 5, wherein the first binding pair member comprises streptavidin and the second binding pair member comprises biotin.

7. The system of claim 1, which comprises (i) a first acceptor bead conjugated to a first chemiluminescent dye and a first anti-idiotype antibody that binds to a first antigen-binding protein in the sample and (ii) a second acceptor bead conjugated to a second chemiluminescent dye and a second anti-idiotype antibody that binds to a second antigen-binding protein in the sample.

8. The system of claim 7, wherein the first chemiluminescent dye comprises terbium (Tb) and the second chemiluminescent dye comprises europium (Eu).

9. The system of claim 7, wherein the first antigen-binding protein is a first therapeutic antibody and the second antigen-binding protein is a second therapeutic antibody.

10. The system of claim 6, which comprises a first conjugate protein comprising the first antigen-binding protein and biotin and a second conjugate protein comprising the second antigen-binding protein and biotin.

11. The system of claim 1, wherein the sample is a composition comprising the two or more different antigen-binding proteins and a pharmaceutically acceptable carrier.

12. A method of determining a presence, absence, or concentration of two or more different antigen-binding proteins in a sample, which method comprises
(1) incubating the sample with the system of claim 1;
(2) exposing the incubated sample to light excitation; and
(3) detecting at least one chemiluminescent signal following light excitation and comparing the detected chemiluminescent signal to a predetermined reference value,
wherein the at least one detected chemiluminescent signal is inversely proportional to the concentration each of the two or more different antigen-binding proteins,
thereby determining the presence, absence, or concentration two or more different antigen-binding proteins in a sample.

13. The method of claim 12, wherein the predetermined reference value comprises a threshold value corresponding to a concentration of each of the antigen-binding proteins, and wherein the detected chemiluminescent signal greater than the threshold value indicates that the sample lacks said antigen-binding protein or comprises said antigen-binding protein below the concentration, and a chemiluminescent signal less than or equal to the threshold value indicates that the sample comprises the antigen-binding protein at a concentration greater than or equal to the concentration.

14. The method of claim 12, wherein the light excitation wavelength is about 500 nm to about 800 nm.

15.-16. (canceled)

17. The method of any one of claim 12, wherein each of the antigen-binding proteins is an antibody, an antibody fragment, or a bispecific T cell engager (BiTE®) molecule.

18. The method of claim 12, wherein the system comprises (i) a first acceptor bead comprising a first chemiluminescent dye and a first anti-idiotype antibody that binds to a first antigen-binding protein in the sample and (ii) a second acceptor bead comprising a second chemiluminescent dye and a second anti-idiotype antibody that binds to a second antigen-binding protein in the sample.

19. The method of claim 18, wherein the first antigen-binding protein comprises a first therapeutic antibody and the second antigen-binding protein comprises a second therapeutic antibody.

20. The method of claim 18, which comprises detecting a first signal emitted from the first chemiluminescent dye and a second signal emitted from the second chemiluminescent dye.

21. The method of claim 20, wherein the first signal and second signal comprise two different wavelengths.

22. The method of claim 21, wherein the wavelength of the first signal is about 520 nm to about 620 nm and the wavelength of the second signal is about 520 nm to about 620 nm.

23. (canceled)

24. The method of claim 12, which further comprises quantifying the two or more antigen-binding proteins in the sample.

25.-26. (canceled)
